(19) United States
(12) Patent Application Publication
(10) Pub. No.: US 2003/0003118 A1
(43) Pub. Date: Jan. 2, 2003

(54) Method and composition for providing skin rejuvenating heat treatment

(76) Inventors: Howard Boilen, Yonkers, NY (US); Adam Lustbader, Chicago, IL (US); Joann Sullivan, Skokie, IL (US); Jose Torres, Chicago, IL (US)

Correspondence Address: R. Neil Sudol, COLEMAN SUDOL SAPONE, P.C., 714 Colorado Avenue, Bridgeport, CT, 06605-1601, US

(21) Appl. No.: 10/084,598

(22) Filed: Feb. 25, 2002

Related U.S. Application Data
(60) Provisional application No. 60/272,257, filed on Feb. 28, 2001.

(51) Int. Cl. A61K 6/00; A61K 7/00; A61K 9/50; A61K 31/20

(52) U.S. Cl. 424/401; 424/502; 514/558

(57) ABSTRACT

A skin rejuvenating heat treatment includes subjecting a hard paraffin wax composition in a microwavable container to microwave energy, thereby melting the wax composition. The melted wax composition is deposited on a human skin surface and subsequently removed from the skin surface. The wax composition includes paraffin wax in an amount of 45% to 85% by weight, water in an amount of 1% to 25% by weight, and surface active componentry including emulsifiers and/or surfactants in an amount of 1% to 54% by weight of the composition.

### CROSS-REFERENCE TO RELATED APPLICATION

This application relies for priority purposes on U.S. provisional application No. 60/272,257 filed Feb. 28, 2001.

### BACKGROUND OF THE INVENTION

This invention relates to a method and to an associated composition for providing a non-invasive skin rejuvenating heat treatment.

It is well known to use paraffin wax to provide heat treatment to certain parts of the body such as hands, feet and elbows. The heat treatment is intended to sooth and condition hands, feet and elbows. The skin is moisturized, thereby reducing dryness of cracks in the treated areas. The treatment also has the effect of relaxing tense muscles, thus easing pain caused by chronic joint inflammation.

Several companies have marketed paraffin bath appliances for home use. These appliances are electrically heated containers provided with built-in electrical resistance heating elements. During home use of such an appliance, paraffin wax blocks are placed into the respective container, which is covered with a lid, and the appliance is plugged into a wall socket. The wax melts, in most cases after a period of several hours, and is heated to a temperature of approximately 130° F. Where a hand is to be treated to moist heat, the hand with the fingers spread is dipped into the melted wax. The hand is gently removed and, after a few seconds during which the wax partially solidifies, the hand is reinserted into the wax melt. This dipping process continues several times. After a short period, the wax is peeled from the hand. The wax may be reused or discarded.

### OBJECTS OF THE INVENTION

An object of the present invention is to provide an improved method for performing a skin rejuvenating heat treatment process of the above-described type.

Another object of the present invention is to provide such a method which may be completed in a shorter period of time than the conventional methods.

It is a further object of the present invention to provide such a method which utilizes a simplified apparatus.

An additional object of the present invention is to provide such a method wherein the apparatus is safer than in the conventional processes.

Yet another object of the present invention is to provide a paraffin wax composition which may be used in such a method.

These and other objects of the present invention will be apparent from the drawings and descriptions herein. It is contemplated that every object is attained by one or more embodiments of the invention. However, not every embodiment need meet every object.

### SUMMARY OF THE INVENTION

A method for providing a skin rejuvenating heat treatment comprises, in accordance with the present invention, placing a substantially predetermined amount wax composition into a microwaveable container, and exposing the container and the wax composition to microwave energy for a period sufficient to heat the wax composition to a predetermined temperature at which the wax composition is liquid. At least a portion of the heated wax composition is subsequently deposited on a human skin surface. After cooling of the deposited wax composition, the cooled deposited wax composition is removed from the skin surface.

Paraffin wax compositions used in the above-described conventional methods for skin rejuvenating heat treatment will not melt if subjected to microwave energy. Thus, it is a surprising and unexpected result to provide a microwave-implemented wax melting process. This result is attained by formulating the wax composition to include wax (preferably but not necessarily paraffin wax), water, and surface active componentry in an amount effective to prevent separation of the water and the wax. More particularly, the microwave-meltable composition used in the method of the present invention includes wax in an amount of 45% to 85% by weight, water in an amount of 1% to 25% by weight, and surface active componentry in an amount of 1% to 54% by weight of the composition.

Pursuant to another feature of the present invention, the surface active componentry includes one or more emulsifying agents. Any emulsifier or group of emulsifiers compatible with paraffin wax and water may be used. Suitable emulsifiers generally include emulsifying waxes (fatty alcohols, fatty acids) and specifically include such emulsifying agents as glyceryl monostearate, cetyl alcohol, stearyl alcohol and propylene glycol. Preferably, but not necessarily, all four of these emulsifying agents are included in a microwavable paraffin wax composition used in a skin rejuvenating heat treatment process in accordance with the invention. Glyceryl monostearate is preferably present in an amount of 2% to 25% by weight of the composition. Cetyl alcohol is preferably present in an amount of 1% to 8% by weight. Stearyl alcohol is preferably included in an amount of 1% to 8% by weight, while propylene glycol is preferably included in an amount of 0.5% to 15% by weight of the composition. The emulsifying componentry together has a weight percentage of 1% to 54% of the paraffin wax composition.

In addition to the emulsifying agents, the surface active componentry may include a surfactant. Preferably, the surfactant is present in an amount of 0.3% to 10% by weight of the paraffin wax composition and is taken from the group consisting of hydrogenated castor oil, alkyl ether sulfates, and alkyl sulfates.

The present invention contemplates a performance of the method in the home. Accordingly, the exposing of the paraffin wax composition to microwave energy includes placing the container with the wax composition into a domestic microwave oven and operating the oven at a predetermined power level. The microwaving of the wax composition may be terminated after a preset period or may be terminated upon a change in appearance of a temperature indicator element mounted to an external surface of the container so as to be visible through a window of an oven door. The temperature indicator is selected to undergo a change in appearance (e.g., color) upon reaching a predetermined temperature. That temperature is in a range between 110° F. and 150° F., preferably between 120° F. and 140° F., and more preferably between 125° F. and 135° F.

As in conventional methods, the heated wax composition is preferably deposited on the skin surface by dipping the skin surface into the liquid wax composition in the container. However, it is possible to use an applicator, such as a brush or a roller to transfer the liquid wax from the container to the skin surface.

The melted wax composition deposited on the skin surface is preferably removed in a conventional manner by peeling the wax layers from the skin surface. However, other methods such as scraping are possible.

Preferably, the wax composition has a pH between about 5.5 and about 8.0.

### BRIEF DESCRIPTION OF THE DRAWINGS

FIG. 1 is a perspective view of a container used in a method in accordance with the present invention.

FIG. 2 is a schematic front elevational view of a microwave oven enclosing the container of FIG. 1, showing a step in a method in accordance with the present invention.

FIG. 3 is a schematic front elevational view of a body portion of the container of FIG. 1, showing a step in the method of the present invention.

### DETAILED DESCRIPTION OF THE DRAWINGS AND DESCRIPTION OF THE PREFERRED EMBODIMENTS

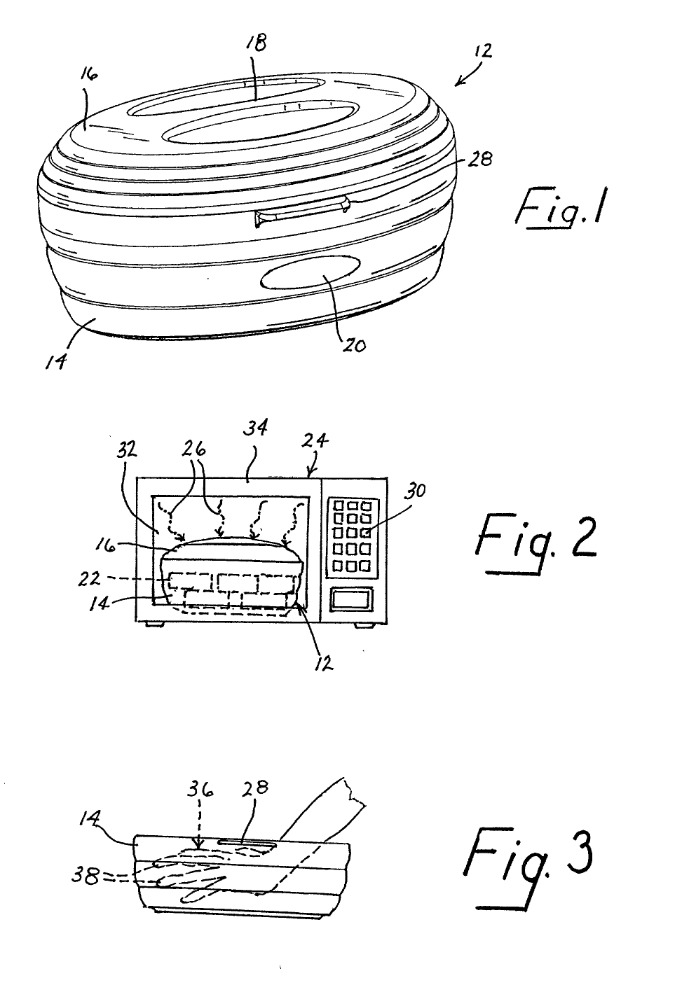

A method for providing a skin rejuvenating heat treatment utilizes a container 12 (FIG. 1) made of a microwavable polymeric material such as polyethylene. Container 12 includes a lower body portion 14 and a lid 16 provided with a handle 18. Container 12 is provided with a temperature indicator element 20 which changes color upon attainment of a predetermined temperature by container 12 and the contents thereof. Indicator element 20 is made of a thermochromic material such as that disclosed in U.S. Pat. No. 6,174,319.

Lid 16 is removed from body portion 14 of container 12. Blocks 22 (FIG. 2) of a paraffin wax composition are placed in the open body portion 14. Then lid 16 is replaced to close the container 12, which is then placed in a countertop or domestic microwave oven 24 (FIG. 2). Microwave energy or radiation 26 generated by oven 24 at a preselected power level converts paraffin wax blocks 22 into a wax melt.

The subjecting of the paraffin wax composition in container 12 to the microwave energy 26 generated by oven 24 may be terminated after a preset period programmed by the user into the oven via a keypad 30. If temperature indicator element 20 has undergone a color change by the end of that preset period, container 12 is removed from oven 24 by using a pair of handles 28 (FIG. 1) secured to lower body portion 14. If the temperature indicator element 20 has not yet changed color, keypad 30 may be operated to set another, generally shorter, time period for the irradiation of container 12 with microwave energy 26. In any case, the subjecting of container 12 and the paraffin wax composition of blocks 22 to microwave energy 26 is terminated no later than a change in the appearance of temperature indicator element 20. Element 20 is mounted to an external surface (not designated) of container 12 so as to be visible through a window 32 of an oven door 34.

Temperature indicator element 20 is made of a material which undergoes a change in color upon reaching a predetermined temperature. That temperature is in a range between 110° F. and 150° F., preferably between 120° F. and 140° F., and more preferably between 125° F. and 135° F. The paraffin wax composition is selected to exist in a liquefied or flowable form within this temperature range. Paraffin waxes having a melting point from 105° F. to 200° F. and more particularly from about 125° F. to about 185° F. are well known in the trade.

After container 12 and the paraffin wax composition of blocks 22 has been exposed to microwave energy 26 for a period sufficient to melt the blocks 22 of paraffin wax composition, container 12 is removed from oven 24. At least a portion of the melted paraffin wax composition is subsequently deposited on a human skin surface. For instance, a user's hand 36 (FIG. 3) preferably with fingers 38 in a spread configuration is dipped into the wax melt in container body portion 14 and then gently removed so that a layer of wax forms on the hand. After a few seconds during which the wax partially solidifies, the hand 36 is reinserted into the wax melt in container lower body portion 14. This dipping process continues several times. After a short period, the wax is peeled from hand 36. The wax may be reused but should be discarded eventually because of contaminant accumulation.

The paraffin wax composition of blocks 22 includes paraffin wax, water, and surface active componentry in an amount effective to prevent separation of the water and the paraffin wax. More particularly, the microwave-meltable composition of blocks 22 includes paraffin wax in an amount of 45% to 85% by weight, water in an amount of 1% to 25% by weight, and surface active componentry in an amount of 1% to 54% by weight of the composition. The water is preferably deionized water.

The surface active componentry includes one or more emulsifiers compatible with paraffin wax and water, such as emulsifying waxes (fatty alcohols, fatty acids). More specifically, the paraffin wax composition includes emulsifying agents as glyceryl monostearate, cetyl alcohol, stearyl alcohol and propylene glycol. Preferred paraffin wax compositions utilizable in a skin rejuvenation heat treatment described above contain all four of these emulsifying agents. Glyceryl monostearate is preferably present in an amount of 2% to 25% by weight of the composition. Cetyl alcohol is preferably present in an amount of 1% to 8% by weight. Stearyl alcohol is preferably included in an amount of 1% to 8% by weight, while propylene glycol is preferably included in an amount of 0.5% to 15% by weight of the composition. The emulsifying componentry together has a weight percentage of 1% to 54% of the paraffin wax composition.

In addition to the emulsifying agents, the surface active componentry may include a surfactant. Preferably, the surfactant is present in an amount of 0.3% to 10% by weight of the paraffin wax composition and is taken from the group consisting of hydrogenated castor oil, alkyl ether sulfates, and alkyl sulfates.

Preferably, the paraffin wax composition has a pH between about 5.5 and about 8.0. The pH may be controlled by well-known agents such as sodium bicarbonate and alpha hydroxy acids. Generally, an acidic pH is more effective for exfoliation of the skin, whereas a higher pH is better for hair removal.

The paraffin wax compositions of the present methodology preferably include skin moisturizers and skin treatment agents such as cocoa butter, avocado oil, methyl paraben and/or propyl paraben. Any other agents suitable for improving the appearance, tone, and resilience of the skin may be incorporated into the wax composition, including, for instance, extracts of almond, aloe vera, apricot, chamomile, peach, rose hips, kola nut, oat bran, orange blossom, cucumber, echinacea, eucalyptus, ginger, ginseng, gotu kola, grapefruit, and haselnut. Additionally or alternatively, suitable extracts of other herbs, fruits, roots, nuts, sea weed, marine extracts, flowers, etc., may be provided in the wax composition.

The paraffin wax compositions of the present methodology also preferably include vitamins such as vitamin C (ascorbic acid) and vitamin E (tocopherol acetate) which are healthful skin treatment agents.

The following is an example of a microwave-meltable paraffin wax composition utilizable in the above-described methodology.

| | |
|---|---|
| Paraffin Wax | 76.05% |
| Glyceryl Monostearate | 7.0% |
| Deionized Water | 7.0% |
| Cetyl Alcohol | 2.5% |
| Stearyl Alcohol | 2.5% |
| Propylene Glycol | 2.0% |
| Peg 40 Hydrogenated | 2.0% |
| Castor Oil | |
| Cocoa Butter | 0.25% |
| Ascorbic Acid (vitamin C) | 0.10% |
| Tocopherol Acetate (vit. E) | 0.10% |
| Avocado Oil | 0.30% |
| Fragrance | 0.10% |
| DMD Hydantoin (and) | 0.10% |
| Methylparaben (and) | |
| Propylparaben | |

The paraffin wax composition of Example 1, when used in the above-described skin rejuvenating heat treatment process, forms a heat glove around the user's hand (or other body part). The wax glove seals in heat and moisture. In addition, the skin conditioning agents such as vitamin C and vitamin E and moisturizers such as cocoa butter and avocado oil are applied to the skin under conditions of heat and moisture, facilitating absorption of those substances into the skin and enhancing the rejuvenating effects.

The paraffin wax composition of Example 1 preferably has a pH of about 7.6 at 124° F. However, that pH level can be varied bin the range of about 5.5 to about 8.5, for instance, depending on whether there is a special need for exfoliation or hair removal.

The following are additional examples of a microwave-meltable paraffin wax composition utilizable in the above-described methodology.

| | |
|---|---|
| Paraffin Wax | 52.0% |
| Glyceryl Monostearate | 10.5% |
| Deionized Water | 23.5% |
| Cetyl Alcohol | 3.75% |
| Stearyl Alcohol | 3.75% |
| Propylene Glycol | 3.0% |
| Peg 40 Hydrogenated | 3.0% |
| Castor Oil | |
| Cocoa Butter | 0.15% |
| Ascorbic Acid | 0.10% |
| Tocopherol Acetate | 0.10% |
| Fragrance | 0.10% |
| DMD Hydantoin (and) | 0.05% |
| Methylparaben (and | |
| Propylparaben | |

The paraffin wax composition of Example 2 is less viscous than the composition of Example 1. This composition results in a thinner layer of wax deposited on a user's hand upon withdrawal of the hand from the reservoir of melted wax. In a typical process using the thinner composition of Example 2, the user inserts the hand or other body part into the wax melt a greater number of times, at a higher repetition rate, than with the composition of Example 1.

The composition of Example 2 is preferable where the principal objective of the user is to relax and sooth underlying muscles and joints, thereby affording pain relief to arthritis sufferers and other individuals with thermally treatable afflictions.

The composition of Example 2 (or any of the compositions disclosed herein) may be rendered more viscous, if desired, by the addition of a thickening agent such as corn starch.

| | |
|---|---|
| Paraffin Wax | 84.0% |
| Glyceryl Monostearate | 3.5% |
| Deionized Water | 4.5% |
| Cetyl Alcohol | 2.0% |
| Stearyl Alcohol | 2.0% |
| Propylene Glycol | 1.5% |
| Peg 40 Hydrogenated | 1.5% |
| Castor Oil | |
| Cocoa Butter | 0.30% |
| Ascorbic Acid | 0.10% |
| Tocopherol Acetate | 0.10% |
| Avocado Oil | 0.30% |
| Fragrance | 0.10% |
| DMD Hydantoin (and) | 0.10% |
| Methylparaben (and | |
| Propylparaben | |

The paraffin wax composition of Example 3 is more viscous than the composition of Example 1. This composition results in a thicker layer of wax deposited on a user's hand as a result of a single insertion of the hand into the reservoir of melted wax. In a typical process using the thicker composition of Example 3, the user inserts the hand or other body part into the wax melt fewer times than with the composition of Example 1. With the composition of Example 3, the time of the overall process is reduced. The heat transferred through the skin of the user from the deposited wax is greater at the beginning of the process than in cases where the initial layer is thin, for example, if the composition of Example 2 is used. The heat transfer later in the process using the composition of Example 3 is much moderated, relative to where the compositions of Example 1 and especially Example 2 are used, owing to the greater insulation effect of the thicker initial layer of wax. The composition of Example 3 is thus preferable where the main or only purpose of the process is cleanse and rejuvenate the skin.

| | |
|---|---|
| Paraffin Wax | 64.0% |
| Glyceryl Monostearate | 8.5% |
| Deionized Water | 10.5% |
| Cetyl Alcohol | 4.0% |
| Stearyl Alcohol | 4.0% |
| Propylene Glycol | 4.0% |
| Peg 40 Hydrogenated | 4.0% |
| Castor Oil | |
| Cocoa Butter | 0.30% |
| Ascorbic Acid | 0.10% |
| Tocopherol Acetate | 0.10% |
| Avocado Oil | 0.30% |
| Fragrance | 0.10% |
| DMD Hydantoin (and) | 0.10% |
| Methylparaben (and | |
| Propylparaben | |

The paraffin wax composition of Example 4 is similar in effect, but less pronounced, than the wax composition of Example 2.

The fragrance used in the microwavable paraffin wax compositions disclosed herein may be selected as desired. Citrus fragrance has proven to be suitable.

Although the invention has been described in terms of particular embodiments and applications, one of ordinary skill in the art, in light of this teaching, can generate additional embodiments and modifications without departing from the spirit of or exceeding the scope of the claimed invention. For instance, the present method may be used in professional salon establishments, as well as in the home.

The wax compositions used in the present invention may use wax other than paraffin wax. Bees wax and other types of wax can be used instead or in addition to paraffin wax.

Although preferred paraffin wax compositions utilizable in carrying out the methods of the present invention include glyceryl monostearate, cetyl alcohol, stearyl alcohol and propylene glycol, one or more of these emulsifying agents may be omitted from the paraffin wax compositions. Moreover, paraffin wax compositions utilizable in a skin rejuvenation heat treatment described herein may instead include other surface contact componentry or emulsifying agents capable of holding water and paraffin wax in a homogenous mixture for purposes of facilitating microwave heating and melting of the wax composition.

The wax deposited into container 12 prior to the application of the microwave energy may take forms other than the blocks disclosed hereinabove. For instance, the unheated wax depositied into container 12 may be in pellet form or even fluid form.

Accordingly, it is to be understood that the drawings and descriptions herein are proffered by way of example to facilitate comprehension of the invention and should not be construed to limit the scope thereof.

What is claimed is:

1. A method for providing a skin rejuvenating heat treatment, comprising:
placing a substantially predetermined amount of a wax composition into a microwavable container;
exposing said container and said wax composition to microwave energy for a period sufficient to heat said wax composition to a predetermined temperature at which said wax composition is liquid;
depositing at least a portion of the heated wax composition on a human skin surface; and
after cooling of the deposited wax composition, removing the cooled deposited wax composition from said skin surface.

2. The method set forth in claim 1 wherein said wax composition includes paraffin wax, water and surface active componentry in an amount effective to prevent separation of the water and the paraffin wax.

3. The method set forth in claim 2 wherein said paraffin wax composition more particularly includes said paraffin wax in an amount of 45% to 85% by weight, said water in an amount of 1% to 25% by weight, and said surface active componentry in an amount of 1% to 54% by weight of said paraffin wax composition.

4. The method set forth in claim 3 wherein said surface active componentry includes emulsifying componentry.

5. The method set forth in claim 4 wherein said emulsifying componentry includes at least one emulsifying agent is taken from the group consisting of glyceryl monostearate, cetyl alcohol, stearyl alcohol and propylene glycol.

6. The method set forth in claim 5 wherein said paraffin wax composition includes glyceryl monostearate in an amount of 2% to 25% by weight of said paraffin wax composition.

7. The method set forth in claim 5 wherein said paraffin wax composition includes cetyl alcohol in an amount of 1% to 8% by weight of said paraffin wax composition.

8. The method set forth in claim 5 wherein said paraffin wax composition includes stearyl alcohol in an amount of 1% to 8% by weight of said paraffin wax composition.

9. The method set forth in claim 5 wherein said paraffin wax composition includes propylene glycol in an amount of 0.5% to 15% by weight of said paraffin wax composition.

10. The method set forth in claim 4 wherein said emulsifying componentry includes at least one an emulsifying wax.

11. The method set forth in claim 4 wherein said emulsifying componentry has a weight percentage of 1% to 54% of said paraffin wax composition.

12. The method set forth in claim 3 wherein said surface active componentry includes a surfactant in an amount of 0.3% to 10% by weight of said paraffin wax composition.

13. The method set forth in claim 12 wherein said surfactant is taken from the group consisting of hydrogenated castor oil, alkyl ether sulfates, and alkyl sulfates.

14. The method set forth in claim 3 wherein the water is deionized water.

15. The method set forth in claim 1 wherein the exposing of said wax composition to microwave energy includes placing said container with the wax composition into a microwave oven and operating said oven at a predetermined power level.

16. The method set forth in claim 15 wherein said container is provided on an outer surface with a temperature indicator element, the exposing of said wax composition to microwave energy further including terminating the operating of said oven upon a change in state of said indicator element indicating attainment of a predetermined temperature by said container and the wax composition disposed therein.

17. The method set forth in claim 1 wherein the depositing of the heated wax composition on said skin surface includes dipping said skin surface into the liquid wax composition in said container.

18. The method set forth in claim 1 wherein the removing of said deposited wax composition includes peeling of said deposited wax composition from said skin surface.

19. The method set forth in claim 1 wherein said wax composition has a pH between about 5.5 and about 8.0.

20. A composition applicable to a skin surface for providing a skin rejuvenating heat treatment, comprising:
wax;
water; and
surface active componentry in an amount effective to prevent separation of the water and the wax.

21. The composition set forth in claim 20, more particularly comprising:
said wax in an amount of 45% to 85% by weight of said composition;
said water in an amount of 1% to 25% by weight of said composition; and
said surface active componentry in an amount of 1% to 54% by weight of said composition.

22. The composition set forth in claim 21 wherein said surface active componentry includes emulsifying componentry.

23. The composition set forth in claim 22 wherein said emulsifying componentry includes at least one emulsifying agent is taken from the group consisting of glyceryl monostearate, cetyl alcohol, stearyl alcohol and propylene glycol.

24. The composition set forth in claim 23 wherein glyceryl monostearate is included in an amount of 2% to 25% by weight of said composition.

25. The composition set forth in claim 25 wherein cetyl alcohol is included in an amount of 1% to 8% by weight of said composition.

26. The composition set forth in claim 24 wherein stearyl alcohol is included in an amount of 1% to 8% by weight of said composition.

27. The composition set forth in claim 24 wherein propylene glycol is included in an amount of 0.5% to 15% by weight of said composition.

28. The composition set forth in claim 22 wherein said emulsifying componentry includes at least one an emulsifying wax.

29. The composition set forth in claim 22 wherein said emulsifying componentry has a weight percentage of 1% to 54% of said composition.

30. The composition set forth in claim 21 wherein said surface active componentry includes a surfactant in an amount of 0.3% to 10% by weight of said composition.

31. The composition set forth in claim 30 wherein said surfactant is taken from the group consisting of hydrogenated castor oil, alkyl ether sulfates, and alkyl sulfates.

32. The composition set forth in claim 21, further comprising at least one skin treatment component taken from the groupd consisting of skin moisurizers, vitamins, botanicals, and extracts.

33. The composition set forth in claim 20 wherein said wax is a paraffin wax.
